(19) United States
(12) Patent Application Publication
(10) Pub. No.: US 2026/0027050 A1
(43) Pub. Date: Jan. 29, 2026

(54) ACOLTREMON COMBINATIONS AND USES THEREOF

(71) Applicant: Alcon Inc., Fribourg (CH)

(72) Inventors: Bhagwati P. Kabra, Euless, TX (US); Joseph Michael Rappon, Southlake, TX (US)

(21) Appl. No.: 19/278,479

(22) Filed: Jul. 23, 2025

Related U.S. Application Data
(60) Provisional application No. 63/674,707, filed on Jul. 23, 2024.

(51) Int. Cl. A61K 9/08; A61K 9/00; A61K 31/167; A61K 31/4725; A61K 38/13; A61K 47/02; A61K 47/38; A61K 47/44; A61P 27/04

(52) U.S. Cl. CPC A61K 9/08; A61K 9/0048; A61K 31/167; A61K 31/4725; A61K 38/13; A61K 47/02; A61K 47/38; A61K 47/44; A61P 27/04

(57) ABSTRACT

Provided herein are combinations comprising: 1) acoltremon; and 2) lifitegrast or cyclosporin, or a pharmaceutically acceptable salt thereof. The combinations are useful for treating dry eye or dysfunctional tear syndrome, or a sign or symptom thereof.

### RELATED APPLICATIONS

This application claims priority of U.S. Provisional Patent Application No. 63/674,707, filed Jul. 23, 2024, the entire content of which is incorporated herein by reference.

### BACKGROUND

Vision is one of the five mammalian sensory perception mechanisms enabling interpretation of and interaction with the world. Interference with vision directly affects an individual's interpretation of and interaction with their world. Eye dryness is very common causing millions of individuals annually to suffer discomfort, red eyes, and other symptoms such as sensitivity to light.

While many options are available to address eye dryness, including dry eye or dysfunctional tear syndrome, there remains a need for improved treatments addressing these therapeutic indications and their signs or symptoms in individuals.

### SUMMARY

Provided herein are combinations of acoltremon with another active pharmaceutical agent, which combinations are useful in treating dry eye.

Also provided herein are methods of administration, comprising ocular instillation of a combination described herein.

Also provided herein are methods of treatment of dry eye in a subject in need thereof, comprising administration of a combination described herein to the subject.

### DETAILED DESCRIPTION

The health and normal functioning of the ocular surface is dependent on a stable and sufficient tear film. The tear film grossly includes three layers: a mucin layer at the corneal/ocular surface; an oil layer at the tear-air interface, and a water layer between the mucin layer and the oil layer. Disruption of any of these layers may result in signs or symptoms of therapeutic indicia, including dry eye or dysfunctional tear syndrome to name a few.

Keratoconjunctivitis sicca, also known as dry eye, xerophthalmia, or keratitis sicca, is dryness of the conjunctiva—a membrane that lines the eyelids and covers the white of the eye—and cornea—a clear layer in front of the iris and pupil. Dry eye may result from dysfunctional tearing such as too few tears produced or too rapid evaporation of tears. Dry eye may also result from deficiency in one or more of the tear layers. For example, dry eye may result from meibomian gland dysfunction resulting in diminished oil layer production. Regardless of its root cause in a given individual, dry eye is very common, causing discomfort, red eyes, and other symptoms such as sensitivity to light in millions of individuals annually. FDA-approved monotherapies are available to address dry eye and include several prescription and over-the-counter ocular instillation solutions as well as intense pulsed light therapy.

Dysfunctional tear syndrome (DTS) is a common and complex condition affecting the ocular surface. DTS may be associated with certain activities. DTS associated with computer or digital screen use, for example, may be characterized by mild irritation, itching, redness, and intermittent tearing after extended staring. It frequently involves foreign body or sandy sensation, blurring of vision, and fatigue, worsening especially at the end of the day. Dry eye and DTS overlap and are frequently comorbid.

Dry eye and DTS may be classified into subtypes including aqueous deficiency, evaporative dry eye, blepharitis/meibomian gland dysfunction (MGD), goblet cell deficiency/mucin deficiency, and exposure-related.

It has been discovered that administration of acoltremon combined with lifitegrast or cyclosporin, or a pharmaceutically acceptable salt thereof, optionally including a semifluorinated alkane (e.g., perfluorohexyloctane or perfluorobutylpentane), effectively address one or more signs or symptoms of dry eye or DTS. In some embodiments, the combinations herein result in a greater population of individuals experiencing improvement of the signs or symptoms of dry eye than either active alone, e.g., a more than additive effect. In some embodiments, the combinations herein improve unstable tear film in subjects in need thereof, e.g., the combinations improve tear film stability as compared to no treatment or as compared to an FDA-approved treatment for dry eye or DTS. Without being bound by theory, administration of acoltremon may lead to a decrease, relative to vehicle alone, in corneal staining (e.g., due to inflammation), and administration of lifitegrast or cyclosporine may lead to reduced inflammation, relative to vehicle alone, and thereby increased tear production. Thus, in some embodiments, the active agents of the combinations herein may act in concert with one another to promote a beneficial cycle within the ocular tissues that addresses more than one sign of dry eye.

Acoltremon (aka, WS-12 or (1R,2S,5R)-2-isopropyl-N-(4-methoxyphenyl)-5-methylcyclohexane-1-carboxamide) is an agonist of transient receptor potential cation channel subfamily M (melastatin) member 8 (TRPM8), which is also known as the cold and menthol receptor 1 (CMR1). The TRPM8 channel is a homotetramer, composed of four identical subunits with a transmembrane domain with six helices (S1-6), and is the primary molecular transducer of cold somatosensation in humans.

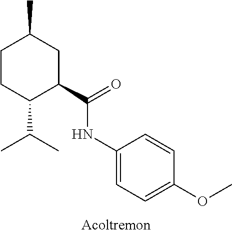 [SMILES: COc1ccc(NC(=O)[C@@H]2C[C@H](C)CC[C@H]2C(C)C)cc1]

Lifitegrast ((S)-2-(2-(benzofuran-6-carbonyl)-5,7-dichloro-1,2,3,4-tetrahydroisoquinoline-6-carboxamido)-3-(3-(methylsulfonyl)phenyl)propanoic acid), an FDA-approved monotherapy for treating the signs and symptoms of dry eye, is an anti-inflammatory agent that binds to the integrin LFA-1, a cell surface protein found on leukocytes and blocks the interaction of LFA-1 with its cognate ligand intercellular adhesion molecule-1 (ICAM-1). ICAM-1 may be overexpressed in corneal and conjunctival tissues in dry eye. LFA-1/ICAM-1 interaction can contribute to the formation of an immunological synapse resulting in T-cell activation and migration to target tissues.

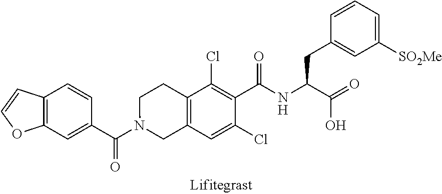 [SMILES: CS(=O)(=O)c1cccc(C[C@H](NC(=O)c2c(Cl)cc3c(c2Cl)CCN(C(=O)c2ccc4ccoc4c2)C3)C(=O)O)c1]

Cyclosporin (cyclo[[(E)-(2S,3R,4R)-3-hydroxy-4-methyl-2-(methylamino)-6-octenoyl]-L-2-aminobutyryl-N-methylglycyl-N-methyl-L-leucyl-L-valylN-methyl-L-leucyl-L-alanyl-D-alanyl-N-methyl-L-leucyl-N-methyl-L-leucyl-N-methyl-L-valyl]), an FDA-approved monotherapy for treating the signs and symptoms of dry eye, is an immunosuppressant. Cyclosporin may increase tear production in patients whose tear production is presumed to be suppressed due to ocular inflammation associated with keratoconjunctivitis sicca.

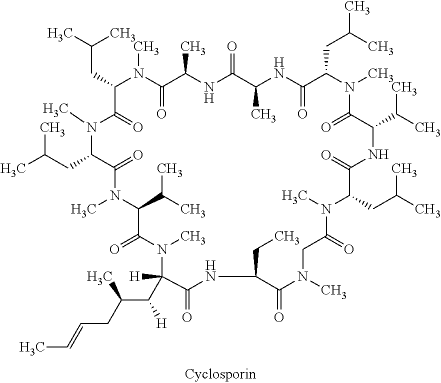

Semifluorinated alkanes, such as perfluorohexyloctane (1,1,1,2,2,3,3,4,4,5,5,6,6-tridecafluorotetradecane), an FDA-approved monotherapy for treating the signs and symptoms of dry eye, may form a monolayer at the air-liquid interface of the tear film which can be expected to reduce evaporation that otherwise leads to dry eye. Perfluorohexyloctane is not soluble in water. Perfluorobutylpentane, another semifluorinated alkane, is included as an inactive ingredient in VEVYE(R) which is another cyclosporine topical ophthalmic formulation for treatment of signs and symptoms of dry eye.

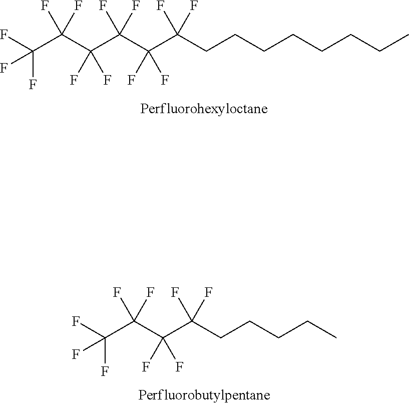 [SMILES: CCCCCCCCC(F)(F)C(F)(F)C(F)(F)C(F)(F)C(F)(F)C(F)(F)F ; CCCCCC(F)(F)C(F)(F)C(F)(F)C(F)(F)F]

### Definitions

Certain terms, whether used alone or as part of a phrase or another term, are defined below.

The articles “a” and “an” refer to one or to more than one of the grammatical object of the article.

Numerical values relating to measurements are subject to measurement errors that place limits on their accuracy. For this reason, the term “about” may explicitly or implicitly modify all numerical values provided herein, unless otherwise indicated.

The term “about” generally indicates a possible variation of a numerical value. In some embodiments, the possible variation (+) may be no more than 10%, 5%, or 1% of the numerical value. In some embodiments, the last decimal place of a numerical value provided herein indicates its degree of accuracy. In some embodiments, where no other error margins are given, the maximum margin is ascertained by applying the rounding-off convention to the last decimal place or last significant digit when a decimal is not present in the given numerical value.

The term “amelioration” means a lessening of severity of at least one indicator of a condition or disease, such as a delay or slowing in the progression of one or more indicators of a condition or disease. The severity of indicators may be determined by subjective or objective measures which are known to those skilled in the art.

The terms “composition” and “pharmaceutical composition” refer to a mixture of at least one compound described herein with another component, such as a carrier or a pharmaceutically acceptable carrier, respectively. The pharmaceutical composition facilitates administration of the compound to a patient or subject.

The terms “effective amount” and “therapeutically effective amount” refer to an amount of therapeutic compound, such as a compound described herein, administered to a subject, either as a single dose or as part of a series of doses, which is effective to produce a desired therapeutic effect.

The term “pharmaceutically acceptable carrier” means a pharmaceutically acceptable material, composition, or carrier, such as a liquid filler, solid filler, stabilizer, dispersing agent, suspending agent, diluent, excipient, thickening agent, solvent, or encapsulating material, involved in carrying or transporting at least one compound described herein within or to the patient such that the compound may perform its intended function. A given carrier must be “acceptable” in the sense of being compatible with the other ingredients of a particular formulation, including the compounds described herein, and not injurious to the patient. Other ingredients that may be included in the pharmaceutical compositions or dosage forms described herein are known in the art and described, for example, in “Remington's Pharmaceutical Sciences” (Genaro (Ed.), Mack Publishing Co., 1985), the entire content of which is incorporated herein by reference.

The term “pharmaceutically acceptable salt” refers to derivatives of the disclosed compounds wherein the parent compound is modified by converting an existing acid or base moiety to its salt form. Lists of salts are found in “Handbook of Pharmaceutical Salts: Properties, Selection, and Use” (P. Henrich Stahl & Camille G. Wermuth (Eds.), VHCA & Wiley-VCH, 2002), the entire content of which is incorporated herein by reference.

The terms “treatment” or “treating” refer to the application of one or more specific procedures used for the amelioration of a disease. A “prophylactic” treatment, refers to reducing the rate of progression of the disease or condition being treated, delaying the onset of that disease or condition, or reducing the severity of its onset.

Recitation of ranges of values herein is merely intended to serve as a shorthand method of referring individually to each separate value falling within the range. Unless otherwise indicated herein, each individual value is incorporated into the specification as if it were individually recited herein. All methods described herein may be performed in any suitable order, and may include a combination of one or more embodiments herein, unless otherwise indicated herein or otherwise clearly contradicted by context. The use of examples, or exemplary language (“for example,” “such as,” etc.) provided herein is intended merely to better illuminate the described subject matter and does not pose a limitation on the scope of the subject matter otherwise claimed. No language in the specification should be construed as indicating any non-claimed element essential to practicing the described subject matter.

Each group member of a grouping of alternative elements or embodiments of this disclosure may be referred to and claimed individually or in any combination with other members of the group or other elements found herein. Furthermore, a recited member of a group may be included in, or excluded from, another recited group for reasons of convenience or patentability.

Reference made to a patent or printed publication document throughout this specification incorporate herein by reference the document's entire content.

Embodiments of this disclosure are illustrative. Accordingly, the present disclosure is not limited to that precisely as shown and described.

### Combinations

Provided herein are combinations (e.g., compositions, e.g., admixtures), comprising 1) acoltremon and 2) lifitegrast or cyclosporin, or a pharmaceutically acceptable salt thereof, the combinations optionally including 3) one or more semifluorinated alkanes (e.g., semifluorinated alkanes independently having a formula C₈₋₁₆H₄₋₂₆F₈₋₁₄). In some embodiments, the combinations herein are admixtures. In some embodiments, the combinations herein are fixed dose combinations. In some embodiments, the acoltremon in the combinations may be present in an amount of about 0.00005% w/v to about 0.006% w/v. In some embodiments, the acoltremon in the combinations may be present in an amount of about 0.0005% w/v to about 0.006% w/v. In some embodiments, the acoltremon in the combinations may be present in an amount of about 0.003% w/v. In some embodiments, the lifitegrast, or a pharmaceutically acceptable salt thereof, in the combinations may be present in an amount of about 1% to about 5% w/v. In some embodiments, the cyclosporin, or a pharmaceutically acceptable salt thereof, in the combinations may be present in an amount of about 0.01 to about 0.15% w/v.

In some embodiments, provided herein are combinations (e.g., compositions, e.g., admixtures) of acoltremon and lifitegrast, or a pharmaceutically acceptable salt thereof. In some embodiments, provided herein are combinations of acoltremon and lifitegrast, or a pharmaceutically acceptable salt thereof, further including one or more compounds having, independently, a formula C₈₋₁₆H₄₋₂₆F₈₋₁₄. In some embodiments, provided herein are combinations of acoltremon and lifitegrast. In some embodiments, the lifitegrast, or its salt, is present in an amount of about 1% to about 5% w/v (e.g., about 10 to about 50 mg/mL).

In some embodiments, the lifitegrast, or its salt, is present in an amount of about 5% w/v (e.g., about 50 mg/mL). In some embodiments, the lifitegrast, or its salt, is present in an amount of about 2% w/v (e.g., about 20 mg/mL). In some embodiments, the lifitegrast, or its salt, is present in an amount of about 1% w/v (e.g., about 10 mg/mL).

In some embodiments, provided herein are combinations (e.g., compositions, e.g., admixtures) of acoltremon and cyclosporin, or a pharmaceutically acceptable salt thereof. In some embodiments, provided herein are combinations of acoltremon and cyclosporin, or a pharmaceutically acceptable salt thereof, further including one or more compounds having, independently, a formula C₈₋₁₆H₄₋₂₆F₈₋₁₄. In some embodiments, provided herein are combinations of acoltremon and cyclosporin. In some embodiments, the cyclosporin, or its salt, is present in an amount of about 0.01 to about 0.15% w/v (e.g., about 0.1 to about 1.5 mg/mL). In some embodiments, the cyclosporin, or its salt, is present in an amount of about 0.01 to about 0.05% w/v (e.g., about 0.1 to about 0.5 mg/mL). In some embodiments, the cyclosporin, or its salt, is present in an amount of about 0.10% w/v (e.g., about 1 mg/mL). In some embodiments, the cyclosporin, or its salt, is present in an amount of about 0.05% w/v (e.g., about 0.5 mg/mL). In some embodiments, the cyclosporin, or its salt, is present in an amount of about 0.03% w/v (e.g., about 0.3 mg/mL).

In some embodiments, the combinations (e.g., compositions, e.g., admixtures) herein are provided as pharmaceutical compositions in a liquid vehicle. In some embodiments, the liquid vehicle is an aqueous vehicle. In some embodiments, the liquid vehicle is a non-aqueous vehicle. In some embodiments, the pharmaceutical compositions comprise less than 1% water.

In some embodiments of the combinations (e.g., compositions, e.g., admixtures) herein, the pharmaceutical compositions or liquid vehicle comprises sodium chloride, dibasic sodium phosphate, sodium thiosulfate, and water. In some embodiments, the pharmaceutical compositions comprise a pH of about 7 to about 8.

In some embodiments of the combinations (e.g., compositions, e.g., admixtures) herein, the pharmaceutical compositions or liquid vehicle comprises glycerin, castor oil, polysorbate 80, carbomer copolymer type A, and water. In some embodiments, the pharmaceutical compositions comprise a pH of about 6.5 to about 8.

In some embodiments of the combinations (e.g., compositions, e.g., admixtures) herein, the pharmaceutical compositions or liquid vehicle comprises polyoxyl 35 castor oil (Kolliphor EL), sodium dihydrogen phosphate (e.g., as sodium dihydrogen phosphate dihydrate), sodium chloride and water, the pharmaceutical compositions comprise a pH of about 6.5 to about 7.5, optionally further including hypromellose (Methocel F4M).

In some embodiments, provided herein are combinations (e.g., compositions, e.g., admixtures), comprising 1) acoltremon and 2) lifitegrast or cyclosporin, or a pharmaceutically acceptable salt thereof.

In some embodiments, the combinations herein comprise acoltremon and lifitregrast or a pharmaceutically acceptable salt thereof.

In some embodiments, the combinations herein comprise acoltremon and cyclosporin or a pharmaceutically acceptable salt thereof.

In some embodiments, the combinations herein are a composition comprising an admixture of the acoltremon and the lifitegrast or cyclosporin, or a pharmaceutically acceptable salt thereof.

In some embodiments, the combinations herein comprise acoltremon and about 1% to about 5% w/v of lifitegrast.

In some embodiments, the combinations herein comprise acoltremon and about 0.01% to about 0.15% w/v of cyclosporin.

In some embodiments, the combinations herein comprise about 0.0005 to 0.006% w/v of acoltremon.

In some embodiments, the combinations herein are a pharmaceutical composition comprising a pharmaceutically acceptable carrier.

In some embodiments, the combinations herein further comprise at least one semifluorinated alkane.

In some embodiments of the compositions herein, a composition comprising acoltremon and lifitegrast comprises about 0.003% w/v acoltremon, about 5% w/v lifitegrast or a salt thereof, about 0.14% hypromellose, about 3.0% w/v polyoxyl 35 castor oil, about 0.78% w/v sodium dihydrogen phosphate (e.g., as a dihydrate), about 0.27% w/v sodium chloride, and may be aqueous (e.g., having water) having a pH of about 7 (which may be achieved by addition of NaOH as needed).

In some embodiments of the compositions herein, a composition comprising acoltremon and lifitegrast comprises about 0.003% w/v acoltremon, about 1% w/v lifitegrast or a salt thereof, about 0.14% hypromellose, about 3.0% w/v polyoxyl 35 castor oil, about 0.78% w/v sodium dihydrogen phosphate (e.g., as a dihydrate), about 0.49% w/v sodium chloride, and may be aqueous (e.g., having water) having a pH of about 7 (which may be achieved by addition of NaOH as needed).

In some embodiments of the compositions herein, a composition comprising acoltremon and cyclosporin comprises about 0.003% w/v acoltremon, about 0.09% w/v cyclosporine or a salt thereof, about 0.14% hypromellose, about 3.0% w/v polyoxyl 35 castor oil, about 0.78% w/v sodium dihydrogen phosphate (e.g., as a dihydrate), about 0.55% w/v sodium chloride, and may be aqueous (e.g., having water) having a pH of about 7 (which may be achieved by addition of NaOH as needed).

In some embodiments of the compositions herein, the composition is selected from Formulation 1, Formulation 2, or Formulation 3.

Herein, reference to combinations may also, in some embodiments, refer to compositions or admixtures.

### Methods

The combinations and compositions herein are useful as medicaments for the signs and symptoms of dry eye or DTS.

The combinations and compositions herein may, in some embodiments, be administered via a variety of routes, including ocular instillation. Accordingly, provided herein are methods of administration, comprising ocular instillation of a combination or pharmaceutical composition described herein.

Thus, in some embodiments, provided herein are methods of treating dry eye in a subject in need thereof, comprising administration of a combination or composition herein to the subject. In some embodiments, provided herein are methods of treating dysfunctional tear syndrome (DTS) in a subject in need thereof, comprising administration of a combination or composition herein to the subject.

In some embodiments, provided herein are methods of treating dry eye or DTS in a subject in need thereof, comprising administration of an admixture of acoltremon and lifitegrast, or a pharmaceutically acceptable salt thereof, to the subject. In some embodiments, the administration is topical administration (e.g., ocular instillation). In some embodiments, the admixture is a pharmaceutical composition. In some embodiments, the pharmaceutical composition further comprises sodium chloride, dibasic sodium phosphate, sodium thiosulfate, and water, optionally having a pH of about 7 to about 8. In some embodiments, the pharmaceutical composition further comprises glycerin, castor oil, polysorbate 80, carbomer copolymer type A, and water, optionally having a pH of about 6.5 to about 8. In some embodiments, the pharmaceutical composition further comprises polyoxyl 35 castor oil (Kolliphor EL), sodium dihydrogen phosphate (e.g., as sodium dihydrogen phosphate dihydrate), sodium chloride and water, the pharmaceutical compositions comprise a pH of about 6.5 to about 7.5, optionally further including hypromellose (Methocel F4M). In some embodiments of these methods, the acoltremon/lifitegrast composition comprises about 0.003% w/v acoltremon, about 1% w/v or 5% w/v lifitegrast or a salt thereof, about 0.14% hypromellose, about 3.0% w/v polyoxyl 35 castor oil, about 0.78% w/v sodium dihydrogen phosphate (e.g., as a dihydrate), about 0.49% w/v or 0.27% w/v sodium chloride, and may be aqueous (e.g., having water) having a pH of about 7 (which may be achieved by addition of NaOH as needed).

In some embodiments, provided herein are methods of treating dry eye or DTS in a subject in need thereof, comprising administration of an admixture of acoltremon and cyclosporin, or a pharmaceutically acceptable salt thereof, to the subject. In some embodiments, the administration is topical administration (e.g., ocular instillation). In some embodiments, the admixture is a pharmaceutical composition. In some embodiments, the pharmaceutical composition further comprises sodium chloride, dibasic sodium phosphate, sodium thiosulfate, and water, optionally having a pH of about 7 to about 8. In some embodiments, the pharmaceutical composition further comprises glycerin, castor oil, polysorbate 80, carbomer copolymer type A, and water, optionally having a pH of about 6.5 to about 8. In some embodiments, the pharmaceutical composition further comprises polyoxyl 35 castor oil (Kolliphor EL), sodium dihydrogen phosphate (e.g., as sodium dihydrogen phosphate dihydrate), sodium chloride and water, the pharmaceutical compositions comprise a pH of about 6.5 to about 7.5, optionally further including hypromellose (Methocel F4M). In some embodiments of these methods, the acoltremon/cyclosporin composition comprises about 0.003% w/v acoltremon, about 0.09% w/v cyclosporine or a salt thereof, about 0.14% hypromellose, about 3.0% w/v polyoxyl 35 castor oil, about 0.78% w/v sodium dihydrogen phosphate (e.g., as a dihydrate), about 0.55% w/v sodium chloride, and may be aqueous (e.g., having water) having a pH of about 7 (which may be achieved by addition of NaOH as needed).

In some embodiments of the methods herein, the combination comprises acoltremon and lifitegrast or a pharmaceutically acceptable salt thereof, and the subject experiences an improvement in a sign or symptom of dry eye or DTS, the improvement being greater than:
1) a first comparative improvement in the sign or symptom of dry eye resulting from corresponding administration of the acoltremon;
2) a second comparative improvement in the sign or symptom of dry eye resulting from corresponding administration of the lifitegrast or a pharmaceutically acceptable salt thereof; or
3) a sum of the improvement of the first comparative improvement and the second comparative improvement.

In some embodiments of the methods herein, the combination comprises acoltremon and cyclosporin or a pharmaceutically acceptable salt thereof, and the subject experiences an improvement in a sign or symptom of dry eye or DTS, the improvement being greater than:
1) a first comparative improvement in the sign or symptom of dry eye resulting from corresponding administration of the acoltremon;
2) a second comparative improvement in the sign or symptom of dry eye resulting from corresponding administration of the cyclosporin or a pharmaceutically acceptable salt thereof; or
3) a sum of the improvement of the first comparative improvement and the second comparative improvement.

In some embodiments of the methods herein, the improvement, the first comparative improvement, and the second comparative improvement are measured by achievement of ≥10 mm increase from baseline in Schirmer Tear Test score.

In some embodiments of the methods herein, the improvement, the first comparative improvement, and the second comparative improvement are measured by reduction of total corneal staining (e.g., fluorescein staining) from baseline.

In some embodiments of the methods herein, the improvement, the first comparative improvement, and the second comparative improvement are measured by reduction in inferior corneal staining (e.g., fluorescein staining) from baseline.

In some embodiments of the methods herein, the improvement, the first comparative improvement, and the second comparative improvement are measured by reduction of total conjunctival staining (e.g., fluorescein, rose bengal, or lissamine green staining) from baseline.

In some embodiments of the methods herein, the improvement of the combinations or compositions (e.g., admixtures) herein (e.g., acoltremon with lifitegrast or acoltremon with cyclosporin) over the individual agent effect is a synergistic effect. Synergy may be determined according to the Colby approach or a generalized Colby approach, e.g., as described in H. Soller and A. Wedemeier, Prediction of synergistic multi-compound mixtures—A generalized Colby approach, Crop Protection, V.42, December 2012, pp. 180-85, which is incorporated herein by reference.

In some embodiments of the methods herein, the composition is selected from Formulation 1, Formulation 2, or Formulation 3.

### Kits

In some embodiments, provided herein are packaged dosage forms, comprising a container holding a therapeutically effective amount of acoltremon and lifitegrast or cyclosporin, or a salt thereof, and instructions for using the dosage form in accordance with one or more of the methods provided herein.

Such dosage forms and associated materials can be finished as a commercial product by the usual steps performed in the present field, for example by appropriate sterilization and packaging steps. For example, the compositions can be sterile filtered. Similarly, the materials can be steam sterilized. Additionally, the packaging materials can be treated by UV/vis irradiation (200-500 nm), for example using photo-initiators with different absorption wavelengths (for example, Irgacure 184, 2959), preferably water-soluble initiators (for example, Irgacure 2959). Such irradiation is usually performed for an irradiation time of 1-60 min, but longer irradiation times may be applied, depending on the specific method. The material according to the present disclosure can be finally sterile-wrapped so as to retain sterility until use and packaged (for example, by the addition of specific product information leaflets) into suitable containers (boxes, etc.). Topical ophthalmic drops for instillation can be packaged in blow fill seal unit dose form vials, or alternatively they may be packaged in a multi-dose preservative free container.

According to further embodiments, the described dosage forms can also be provided in kit form combined with other components necessary for administration of the material to the patient. For example, disclosed kits, such as for use in the treatments described herein, can further comprise, for example, administration materials.

The kits may be designed in various forms based on the specific deficiencies they are designed to treat.

When the dosage forms provided herein are stored in a polyolefin plastic container (e.g., low density polyethylene (LDPE) or high density polyethylene (HDPE)) as compared to, for example, a polyvinyl chloride plastic container, discoloration or cloudiness of the dosage form may be comparatively less. Without being bound by theory, the container may reduce exposure of the container's contents to electromagnetic radiation, whether visible light (for example, having a wavelength of about 380-780 nm) or ultraviolet (UV) light (for example, having a wavelength of about 190-320 nm (UV B light) or about 320-380 nm (UV A light)). Some containers also include the capacity to reduce adherence or adsorption of the active ingredient to the surface of the container, which could effectively dilute the concentration of active ingredient in the contained solution. Some containers also include the capacity to reduce exposure of the container's contents to infrared light, or a second component with such a capacity. Some containers further include the capacity to reduce the exposure of the container's contents to heat or humidity. The containers that may be used include those made from a polyolefin such as polyethylene, polypropylene, polyethylene terephthalate, polycarbonate, polymethylpentene, polybutene, or a combination thereof, especially polyethylene, polypropylene, or a combination thereof. In some embodiments, the container is a glass container. The container may further be disposed within a second container, for example, a paper container, cardboard container, paperboard container, metallic film container, or foil container, or a combination thereof, to further reduce exposure of the container's contents to UV, visible, or infrared light. The dosage forms provided herein may need storage lasting up to, or longer than, three months; in some cases, up to, or longer than one year. The containers may be in any form suitable to contain the contents—for example, a bag, a bottle, or a box.

### EXAMPLES

The following examples further illustrate embodiments of the present disclosure.

However, they are in no way a limitation of the teachings or disclosure as described herein.

### Example 1: Stability of 0.003% Acoltremon 0.09% Cyclosporine Ophthalmic Solution (Formulation 1)

A combination formulation of 0.003% Acoltremon and 0.09% Cyclosporine of the following composition (exemplary Formulation 1) is prepared and tested for stability of active components at 5° C., 25° C., and/or 40° C. The results are acceptable for long-term (e.g., 3-month, 12-month, 18-month, 24-month, 36-month, or longer) storage of the formulation at 5° C.

TABLE 1
Exemplary Formulation 1.

| Component | Concentration w/v % |
|---|---|
| Acoltremon | 0.003 |
| Cyclosporine | 0.09 |
| Hypromellose (Methocel F4M) | 0.14 |
| Polyoxyl 35 Castor Oil (Kolliphor EL) | 3.0 |
| Sodium Dihydrogen Phosphate Dihydrate | 0.78 |
| Sodium Chloride | 0.55 |
| Sodium Hydroxide, | q.s. to pH 7 |
| Purified Water | q.s. to 100 mL |

### Example 2: Ocular Tissue Distribution of 0.003% Acoltremon 0.09% Cyclosporine Ophthalmic Solution (Formulation 1)

Solutions of a combination herein are prepared and assayed in rabbits to determine their distribution throughout ocular tissue following ocular instillation and is compared to 0.003% Acoltremon ophthalmic solution, 0.09% cyclosporine (Cequa®) ophthalmic solution or 0.05% Cyclosporine (Restasis®) ophthalmic solution. The results show similar levels of acoltremon in cornea between the combination and 0.003% Acoltremon ophthalmic solution. The results also show similar levels of cyclosporine in cornea between the combination and 0.09% cyclosporine (Cequa®) ophthalmic solution or 0.05% Cyclosporine (Restasis®) ophthalmic solution.

### Example 3: Clinical Testing of 0.003% Acoltremon 0.09% Cyclosporine Ophthalmic Solution (Formulation 1)

In a clinical study, patients with dry eye are randomized to 0.003% Acoltremon ophthalmic solution, 0.09% cyclosporine (Cequa®) ophthalmic solution or 0.05% Cyclosporine (Restasis®) ophthalmic solution, and 0.003% Acoltremon 0.09% cyclosporine ophthalmic solution twice daily for 12 weeks. Patient eligibility criteria are DED signs and symptoms of prespecified severity levels. Outcomes assessments include DED signs ≥10 mm Improvement from Baseline in Schirmer's Tear Test Score, total corneal staining, inferior corneal staining, and total conjunctival staining at 2 weeks, 4 weeks, 6 weeks, 8 weeks, and/or 12 weeks timepoints.

The outcome is that the combination shows superior DED sign, namely one or more of:
1) ≥10 mm Improvement from Baseline in Schirmer's Tear Test Score compared to individual monotherapies at one or more of the timepoints tested;
2) reduction of total corneal staining from baseline compared to individual monotherapies at one or more of the timepoints tested;
3) reduction in inferior corneal staining from baseline compared to individual monotherapies at one or more of the timepoints tested; or
4) reduction of total conjunctival staining from baseline compared to individual monotherapies at one or more of the timepoints tested.

### Example 4: Clinical Testing of 0.003% Acoltremon 0.09% Cyclosporine Ophthalmic Solution (Formulation 1)

In a clinical study, patients with dry eye are randomized to 0.003% Acoltremon ophthalmic solution, 0.09% cyclosporine (Cequa®) ophthalmic solution or 0.05% Cyclosporine (Restasis®) ophthalmic solution, and 0.003% Acoltremon 0.09% cyclosporine ophthalmic solution twice daily for 12 weeks. Patient eligibility criteria are DED signs and symptoms of prespecified severity levels. Outcomes assessments include DED signs ≥10 mm Improvement from Baseline in Schirmer's Tear Test Score, total corneal staining, inferior corneal staining, and total conjunctival staining at 2 weeks, 4 weeks, 6 weeks, 8 weeks, and/or 12 weeks timepoints.

The outcome is that the combination shows synergistic DED sign, namely one or more of:
1) ≥10 mm Improvement from Baseline in Schirmer's Tear Test Score compared to individual monotherapies at one or more of the timepoints tested;
2) reduction of total corneal staining from baseline compared to individual monotherapies at one or more of the timepoints tested;
3) reduction in inferior corneal staining from baseline compared to individual monotherapies at one or more of the timepoints tested; or
4) reduction of total conjunctival staining from baseline compared to individual monotherapies at one or more of the timepoints tested.

### Example 5: Stability of 0.003% Acoltremon 5% Lifitegrast Ophthalmic Solution (Formulation 2)

A combination formulation of 0.003% Acoltremon and 5% Lifitegrast of the following composition (exemplary Formulation 2) is prepared and tested for stability of active components at 5° C., 25° C., and/or 40° C. The results are acceptable for long-term (e.g., 3-month, 12-month, 18-month, 24-month, 36-month, or longer) storage of the formulation at 5° C.

TABLE 2
Exemplary Formulation 2.

| Component | Concentration w/v % |
|---|---|
| Acoltremon | 0.003 |
| Lifitegrast | 5 |
| Hypromellose (Methocel F4M) | 0.14 |
| Polyoxyl 35 Castor Oil (Kolliphor EL) | 3.0 |
| Sodium Dihydrogen Phosphate Dihydrate | 0.78 |
| Sodium Chloride | 0.27 |
| Sodium Hydroxide, | q.s. to pH 7 |
| Purified Water | q.s. to 100 mL |

### Example 6: Ocular Tissue Distribution of 0.003% Acoltremon 5% Lifitegrast Ophthalmic Solution (Formulation 2)

Solutions of a combination herein are prepared and assayed in rabbits to determine their distribution throughout ocular tissue following ocular instillation and is compared to 0.003% Acoltremon ophthalmic solution, and 5% Lifitegrast ophthalmic solution (Xiidra). The results show similar levels of acoltremon in cornea between the combination and 0.003% Acoltremon ophthalmic solution. The results also show similar levels of lifitegrast in cornea between the combination and 5% Lifitegrast ophthalmic solution (Xiidra).

### Example 7: Clinical Testing of 0.003% Acoltremon 5% Lifitegrast Ophthalmic Solution (Formulation 2)

In a clinical study, patients with dry eye are randomized to 0.003% Acoltremon ophthalmic solution, 5% Lifitegrast ophthalmic solution (Xiidra), and 0.003% Acoltremon 5% Lifitegrast ophthalmic solution twice daily for 12 weeks. Patient eligibility criteria are DED signs and symptoms of prespecified severity levels. Outcomes assessments include DED signs ≥10 mm Improvement from Baseline in Schirmer's Tear Test Score, total corneal staining, inferior corneal staining, and total conjunctival staining at 2 weeks, 4 weeks, 6 weeks, 8 weeks, and/or 12 weeks timepoints.

The outcome is that the combination shows superior DED sign, namely one or more of:
1) ≥10 mm Improvement from Baseline in Schirmer's Tear Test Score compared to individual monotherapies at one or more of the timepoints tested;
2) reduction of total corneal staining from baseline compared to individual monotherapies at one or more of the timepoints tested;
3) reduction in inferior corneal staining from baseline compared to individual monotherapies at one or more of the timepoints tested; or
4) reduction of total conjunctival staining from baseline compared to individual monotherapies at one or more of the timepoints tested.

### Example 8: Clinical Testing of 0.003% Acoltremon 5% Lifitegrast Ophthalmic Solution (Formulation 2)

In a clinical study, patients with dry eye are randomized to 0.003% Acoltremon ophthalmic solution, 5% Lifitegrast ophthalmic solution (Xiidra), and 0.003% Acoltremon 5% Lifitegrast ophthalmic solution twice daily for 12 weeks. Patient eligibility criteria are DED signs and symptoms of prespecified severity levels. Outcomes assessments include DED signs ≥10 mm Improvement from Baseline in Schirmer's Tear Test Score, total corneal staining, inferior corneal staining, and total conjunctival staining at 2 weeks, 4 weeks, 6 weeks, 8 weeks, and/or 12 weeks timepoints.

The outcome is that the combination shows synergistic DED sign, namely one or more of:
1) ≥10 mm Improvement from Baseline in Schirmer's Tear Test Score compared to individual monotherapies at one or more of the timepoints tested;
2) reduction of total corneal staining from baseline compared to individual monotherapies at one or more of the timepoints tested;
3) reduction in inferior corneal staining from baseline compared to individual monotherapies at one or more of the timepoints tested; or
4) reduction of total conjunctival staining from baseline compared to individual monotherapies at one or more of the timepoints tested.

### Example 9: Stability of 0.003% Acoltremon 1% Lifitegrast Ophthalmic Solution (Formulation 3)

A combination formulation of 0.003% Acoltremon and 1% Lifitegrast of the following composition (exemplary Formulation 3) is prepared and tested for stability of active components at 5° C., 25° C., and/or 40° C. The results are acceptable for long-term (e.g., 3-month, 12-month, 18-month, 24-month, 36-month, or longer) storage of the formulation at 5° C.

TABLE 3
Exemplary Formulation 3.

| Component | Concentration w/v % |
|---|---|
| Acoltremon | 0.003 |
| Lifitegrast | 1 |
| Hypromellose (Methocel F4M) | 0.14 |
| Polyoxyl 35 Castor Oil (Kolliphor EL) | 3.0 |
| Sodium Dihydrogen Phosphate Dihydrate | 0.78 |
| Sodium Chloride | 0.49 |
| Sodium Hydroxide, | q.s. to pH 7 |
| Purified Water | q.s. to 100 mL |

### Example 10: Ocular Tissue Distribution of 0.003% Acoltremon 1% Lifitegrast Ophthalmic Solution (Formulation 2)

Solutions of a combination herein are prepared and assayed in rabbits to determine their distribution throughout ocular tissue following ocular instillation and is compared to 0.003% Acoltremon ophthalmic solution, and 1% Lifitegrast ophthalmic solution (in Xiidra vehicle). The results show similar levels of acoltremon between the combination and 0.003% Acoltremon ophthalmic solution. The results also show similar levels of lifitegrast between the combination and 1% Lifitegrast ophthalmic solution (in Xiidra vehicle).

### Example 11: Clinical Testing of 0.003% Acoltremon 1% Lifitegrast Ophthalmic Solution (Formulation 3)

In a clinical study, patients with dry eye are randomized to 0.003% Acoltremon ophthalmic solution, 5% Lifitegrast ophthalmic solution (Xiidra), and 0.003% Acoltremon 1% Lifitegrast ophthalmic solution twice daily for 12 weeks. Patient eligibility criteria are DED signs and symptoms of prespecified severity levels. Outcomes assessments include DED signs ≥10 mm Improvement from Baseline in Schirmer's Tear Test Score, total corneal staining, inferior corneal staining, and total conjunctival staining at 2 weeks, 4 weeks, 6 weeks, 8 weeks, and/or 12 weeks timepoints.

The outcome is that the combination shows superior DED sign, namely one or more of:
1) ≥10 mm Improvement from Baseline in Schirmer's Tear Test Score compared to individual monotherapies at one or more of the timepoints tested;
2) reduction of total corneal staining from baseline compared to individual monotherapies at one or more of the timepoints tested;
3) reduction in inferior corneal staining from baseline compared to individual monotherapies at one or more of the timepoints tested; or
4) reduction of total conjunctival staining from baseline compared to individual monotherapies at one or more of the timepoints tested.

### Example 12: Clinical Testing of 0.003% Acoltremon 1% Lifitegrast Ophthalmic Solution (Formulation 3)

In a clinical study, patients with dry eye are randomized to 0.003% Acoltremon ophthalmic solution, 5% Lifitegrast ophthalmic solution (Xiidra), and 0.003% Acoltremon 1% Lifitegrast ophthalmic solution twice daily for 12 weeks. Patient eligibility criteria are DED signs and symptoms of prespecified severity levels. Outcomes assessments include DED signs ≥10 mm Improvement from Baseline in Schirmer's Tear Test Score, total corneal staining, inferior corneal staining, and total conjunctival staining at 2 weeks, 4 weeks, 6 weeks, 8 weeks, and/or 12 weeks timepoints.

The outcome is that the combination shows synergistic DED sign, namely one or more of:
1) ≥10 mm Improvement from Baseline in Schirmer's Tear Test Score compared to individual monotherapies at one or more of the timepoints tested;
2) reduction of total corneal staining from baseline compared to individual monotherapies at one or more of the timepoints tested;
3) reduction in inferior corneal staining from baseline compared to individual monotherapies at one or more of the timepoints tested; or
4) reduction of total conjunctival staining from baseline compared to individual monotherapies at one or more of the timepoints tested.

1. A combination, comprising 1) acoltremon and 2) lifitegrast or cyclosporin, or a pharmaceutically acceptable salt thereof.

2. The combination of claim 1, comprising acoltremon and lifitegrast.

3. The combination of claim 1, comprising acoltremon and cyclosporin.

4. The combination of claim 1, which is a composition comprising an admixture of the acoltremon and the lifitegrast or cyclosporin, or a pharmaceutically acceptable salt thereof.

5. The combination of claim 4, comprising acoltremon and about 1% to about 5% w/v of lifitegrast.

6. The combination of claim 4, comprising acoltremon and about 0.01% to about 0.15% w/v of cyclosporin.

7. The combination of one of claims 1-6, comprising about 0.0005 to 0.006% w/v of acoltremon.

8. (canceled)

9. The combination of claim 1, further comprising at least one semifluorinated alkane.

10. The combination of claim 1, which is a composition, comprising:
about 0.003% w/v acoltremon,
about 5% w/v lifitegrast or a salt thereof,
about 0.14% w/v hypromellose,
about 3.0% w/v polyoxyl 35 castor oil,
about 0.78% w/v of a sodium dihydrogen phosphate, and
about 0.27% w/v sodium chloride;
and, optionally, wherein the composition further comprises one of more of water, a pH of about 7, and NaOH.

11. The combination of claim 1, which is a composition, comprising:
about 0.003% w/v acoltremon,
about 1% w/v lifitegrast or a salt thereof,
about 0.14% w/v hypromellose,
about 3.0% w/v polyoxyl 35 castor oil,
about 0.78% w/v of a sodium dihydrogen phosphate, and
about 0.49% w/v sodium chloride;
and, optionally, wherein the composition further comprises one of more of water, a pH of about 7, and NaOH.

12. The combination of claim 1, which is a composition, comprising:
about 0.003% w/v acoltremon,
about 0.09% w/v cyclosporine or a salt thereof,
about 0.14% w/v hypromellose,
about 3.0% w/v polyoxyl 35 castor oil,
about 0.78% w/v of a sodium dihydrogen phosphate, and
about 0.55% w/v sodium chloride;
and, optionally, wherein the composition further comprises one of more of water, a pH of about 7, and NaOH.

13. (canceled)

14. A method of administration, comprising administration of the combination of claim 1 to an eye of a subject by ocular instillation.

15. A method of treating a sign or symptom of dry eye or dysfunctional tear syndrome in a subject in need thereof, comprising administration of the combination of claim 1 to an eye of the subject by ocular instillation.

16. A method of treating dry eye or dysfunctional tear syndrome in a subject in need thereof, comprising administration of the combination of claim 1 to an eye of the subject by ocular instillation.

17. The method of claim 15, wherein the combination comprises acoltremon and lifitegrast or a pharmaceutically acceptable salt thereof, or the combination comprises acoltremon and cyclosporin or a pharmaceutically acceptable salt thereof, and the subject experiences an improvement in a sign or symptom of dry eye, the improvement being greater than:
1) a first comparative improvement in the sign or symptom of dry eye resulting from corresponding administration of the acoltremon;
2) a second comparative improvement in the sign or symptom of dry eye resulting from corresponding administration of the lifitegrast or a pharmaceutically acceptable salt thereof; or
3) a sum of the improvement of the first comparative improvement and the second comparative improvement.

18. (canceled)

19. The method of claim 17, wherein the improvement, the first comparative improvement, and the second comparative improvement are measured by achievement of ≥10 mm increase from baseline in Schirmer Tear Test score.

20. The method of claim 17, wherein the improvement, the first comparative improvement, and the second comparative improvement are measured by reduction in total corneal staining from baseline

21. The method of claim 17, wherein the improvement, the first comparative improvement, and the second comparative improvement are measured by reduction in inferior corneal staining from baseline

22. The method of claim 17, wherein the improvement, the first comparative improvement, and the second comparative improvement are measured by reduction of total conjunctival staining from baseline.

23. The method of claim 17, wherein the improvement, the first comparative improvement, and the second comparative improvement are measured by reduction in corneal staining as well as by achievement of ≥10 mm increase from baseline in Schirmer Tear Test score.
